(19) United States
(12) Patent Application Publication
(10) Pub. No.: US 2021/0112965 A1
(43) Pub. Date: Apr. 22, 2021

(54) SYSTEMS AND METHODS FOR PROVIDING PERSONALIZED ORAL CARE FEEDBACK TO A USER

(71) Applicant: KONINKLIJKE PHILIPS N.V., EINDHOVEN (NL)

(72) Inventors: Vincent JEANNE, Migne Auxances (FR); Chong Bing WONG, Bothell, WA (US)

(21) Appl. No.: 17/047,448

(22) PCT Filed: Apr. 18, 2019
(86) PCT No.: PCT/EP2019/060032

§ 371 Date: Oct. 14, 2020

Related U.S. Application Data
(60) Provisional application No. 62/660,316, filed on Apr. 20, 2018.

(51) Int. Cl. A46B 15/00; A46B 17/00

(52) U.S. Cl. CPC A46B 15/0044; A46B 2200/1066; A46B 17/00

(57) ABSTRACT

An oral care system and method for providing visual feedback to a user after usage sessions of an oral care device. The system includes a sensor module configured to collect data during usage sessions with the oral care device related to a longitudinal parameter representative of an oral health of a user. A memory module is configured to store the data. A data interpretation module is configured to retrieve historical usage data from the memory module and quantify oral health values from the historical data with respect to each of the longitudinal parameters. A decision module is configured to compare the oral health values to a set of reference values. A controller is configured to generate a control signal based on an output of the decision module. A visual feedback module of the oral care device is configured to operate in accordance with the control signal.

### FIELD OF THE INVENTION

The present disclosure is directed generally to oral care systems and methods, more specifically systems and methods that provide personalized oral care feedback to users.

### BACKGROUND

Acquiring and maintaining good oral care habits is important to both oral and systemic health. However, despite numerous recommendations from health professionals, dedicated coaching via smart oral care products (e.g., networked or internet connected toothbrushes), complying with an oral care regimen remains a challenge for many consumers. For example, the impact of professional recommendation often fades away in a short period of time after visiting a dental office, and currently available smart oral care products require the users of these devices to perform extra procedures or dedicated actions unrelated to the oral care itself (e.g., use an application installed on a smartphone paired to their oral care device).

Accordingly, there is a continued need in the art for oral care systems and methods that facilitate good oral care habits in users.

### SUMMARY OF THE INVENTION

The present disclosure is directed to inventive oral care systems and methods for providing personalized feedback to a user. An oral care system may include an oral care device configured to provide one or more oral care functions for a user (e.g., a brushing, flossing, oral irrigation, etc.). The oral care device also includes a visual feedback module, such as one or more light sources, configured to operate in accordance with different operational characteristics (e.g., colors, patterns, symbols, etc.) in response to a control signal sent by a controller.

The controller includes one or more sensor modules configured to collect data related to one or more longitudinal parameters representative or indicative of the user's oral health while using oral care device, such as coverage, session duration, daily/weekly use count, applied brushing force, etc. One or more values representative of the oral health are quantified from the collected data. The quantified oral health values are compared for conformity or compliance with a set of one or more reference values (e.g., it is determined whether the quantified oral health values fall within expected ranges or above/below expected thresholds indicative of good oral health and/or compliance with a selected oral care regimen). The control signal for the visual feedback module may be generated based on the degree of conformity with the reference values. Advantageously, the operation of the visual feedback module is automatically updated after each usage session, which enables users to quickly and consistently view a snapshot or overview of their oral health or oral care habits without disrupting of their existing oral care routine with extra procedures or actions unrelated to the oral care itself.

Generally in one aspect, an oral care system is provided. The oral care system includes: an oral care device; a sensor module configured to collect data during usage sessions with the oral care device related to one or more longitudinal parameters representative of oral health of a user; a memory module configured to store the data; a data interpretation module configured to retrieve historical usage data from the memory module and quantify one or more oral health values from the historical usage data with respect to each of the one or more longitudinal parameters; a decision module configured to compare the one or more health values to a set of one or more reference values; and a controller configured to generate a control signal based on an output of the decision module; and a visual feedback module of the oral care device configured to operate in accordance with the control signal.

According to an embodiment, the one or more longitudinal parameters includes a plurality of longitudinal parameters, the one or more oral health values includes a plurality of oral health values, and the data interpretation module is configured to quantify one of the oral health values for each of the longitudinal parameters.

According to an embodiment, the controller is configured to generate a plurality of control signals, with one of the control signals generated with respect to each of the oral health values, and the visual feedback module includes separate elements that are independently controllable with each of the control signals.

According to an embodiment, the visual feedback module includes one or more light sources.

According to an embodiment, the control signal instructs the one or more light sources to be operated with a first set of operating characteristics if the output of the decision module relates to a sufficient degree of conformity of the one or more oral health values to the set of one or more reference values and to be operated with a second set of operating characteristics if the output of the decision module relates to an insufficient degree of conformity of the one or more oral health values to the set of one or more reference values.

According to an embodiment, the first and second operating characteristics include different colors, patterns, intensities, number of illuminated light sources, or a combination including at least one of the foregoing.

According to an embodiment, the set of reference values includes at least one threshold value or range of values.

According to an embodiment, data collected during each usage session is arranged in a dataset associated with that usage session.

According to an embodiment, the one or more oral health values comprise a plurality of oral health values, and the controller is configured to quantify one of the oral health values for each of the datasets.

According to an embodiment, the historical usage data includes a most recent usage session and at least one previous usage session aggregated together.

According to an embodiment, the system further comprises a remote device in communication with the oral care device, the remote device at least partially comprising the controller.

According to an embodiment, the oral care device includes a main body, a workpiece, a charging station, or a combination including at least one of the foregoing.

Generally in one aspect, an oral care device is provided. The oral care device includes: a sensor module configured to collect data during usage sessions with the oral care device related to a longitudinal parameter representative of oral health of a user; a memory module configured to store the data; a controller configured to, after each usage session of the oral care device, automatically: retrieve the data for a most recent usage session and at least one previous usage session; quantify an oral health value of the longitudinal parameter from the retrieved data; perform a comparison of the oral health value to a set of one or more reference values; and generate a control signal based on the comparison; and a visual feedback module of the oral care device configured to operate in accordance with the control signal.

Generally in one aspect, a method for providing visual feedback to a user of an oral care device is provided. The method includes: collecting data during a usage session of the oral care device, the data related to a longitudinal parameter representative of oral health of the user; storing the data in a memory module; retrieving historical usage data from the memory module; quantifying an oral health value of the longitudinal parameter from the retrieved data; comparing the oral health value to a set of one or more reference values; generating a control signal based on the comparing; and operating a visual feedback module of the oral care device in accordance with the control signal; wherein one or more of the receiving, quantifying, comparing, and generating occur automatically after usage sessions of the oral care device.

According to an embodiment, the generating includes generating a plurality of control signals, the visual feedback module includes a plurality of separate elements, and the operating includes separately controlling each of separate elements of the visual feedback module in accordance with a different one of the control signals

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 schematically illustrates an oral care system according to one embodiment disclosed herein.

FIG. 2 is a front view of a main body of an oral care device having a visual feedback module according to one embodiment disclosed herein.

FIG. 3 is a block diagram illustrating a controller for an oral care system according to one embodiment disclosed herein.

FIG. 4 is a graph illustrating quantified oral health values determinable by a data interpretation module for a number of usage sessions according to one embodiment disclosed herein.

FIG. 5 illustrates a visual feedback module having a plurality of independent elements that are separately controllable.

### DETAILED DESCRIPTION OF EMBODIMENTS

The present disclosure describes various embodiments of systems and methods for automatically assessing a user's oral care usage sessions and providing visual feedback regarding the user's oral health over time. More generally, Applicant has recognized and appreciated that it would be beneficial to provide a system and method for providing personalized feedback to the user's oral health and habits. A particular goal of utilization of certain embodiments of the present disclosure is to automatically provide users with feedback regarding their oral health over time without requiring the user to perform extra procedures or dedicated actions unrelated to the oral care itself.

In view of the foregoing, various embodiments and implementations are directed to oral care systems and methods for providing personalized feedback to a user. An oral care system may include an oral care device configured to provide one or more oral care functions for a user (e.g., brushing, flossing, oral irrigation, etc.). The oral care device also includes a visual feedback module, such as one or more light sources, configured to operate in accordance with different operational characteristics (e.g., colors, patterns, symbols, etc.) in response to a control signal sent by a controller.

The controller includes one or more sensor modules configured to collect data related to one or more longitudinal parameters representative or indicative of the user's oral health while using oral care device, such as coverage, session duration, daily/weekly use count, applied brushing force, etc. One or more values representative of the oral health are quantified from the collected data. The quantified oral health values are compared for conformity or compliance with a set of one or more reference values (e.g., it is determined whether the quantified oral health values fall within expected ranges or above/below expected thresholds indicative of good oral health and/or compliance with a selected oral care regimen). The control signal for the visual feedback module may be generated based on the degree of conformity with the reference values. Advantageously, the operation of the visual feedback module is automatically updated after each usage session, which enables users to quickly and consistently view a snapshot or overview of their oral health and habits without disrupting of their existing oral care routine with extra procedures or actions unrelated to the oral care itself.

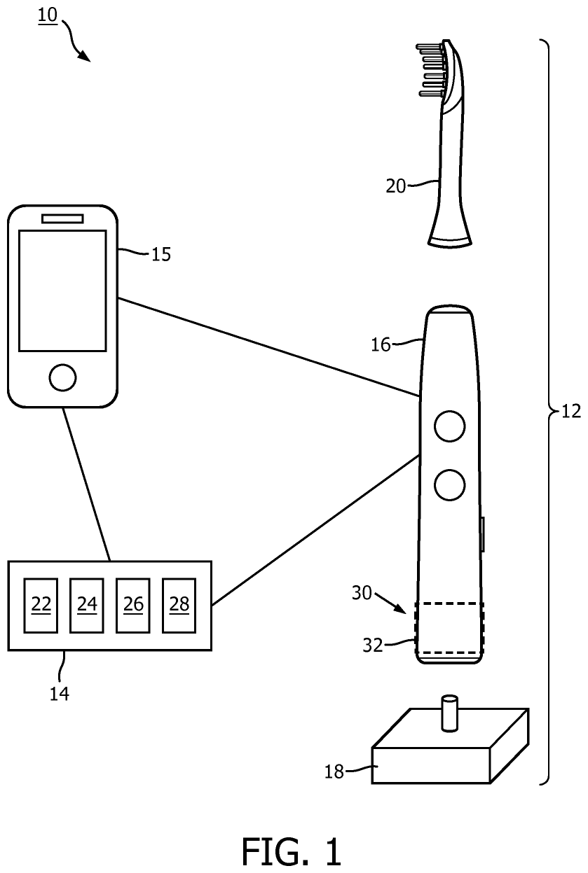

Referring to FIG. 1, an oral care system 10 illustrated having an oral care device 12, a controller 14, and a remote device 15. The oral care device 12 includes a main body 16 and may additionally include a charging station (or base) 18, and/or a workpiece 20. In the illustrated embodiment, the oral care device 12 takes the form of an electric toothbrush assembly, with the workpiece 20 provided as a brush head that is removably attachable to the main body 16. In this example, the main body 16 may include a motor, drive train, battery, and other software and hardware elements configured to cause pre-programmed operation of the main body 16 and/or the workpiece 20 in response to user inputs, e.g., to cause oscillations or other brushing motions of the brush head when the oral care device 12 is activated by a power button on the main body 16. It is to be appreciated that in other embodiments, the oral care device 12 may be an oral irrigator, an electric flossing device, or other oral care device. The workpiece 20 may take a variety of different forms that are permanently attached or removably attachable to the main body 16 to enable various different oral care procedures or functionality (e.g., the workpiece 20 may include a flossing or irrigation nozzle, a tongue cleaner, a toothbrush head, or a combination of one or more of these functions, etc.).

The controller 14 may be included in any combination of components of the system 10, e.g., the oral care device 12 (including any of the main body 16, the charging station 18, the workpiece 20), the remote device 15, etc. For example, the oral care device 12 and the remote device 15 may include separate controllers or computing resources that together form the controller 14 as discussed herein. In other words, computing components can be considered part of the controller 14 regardless of whether or which component of the system 10 those computing components are embedded in or arranged in associated with.

The controller 14 may include a processor 22, a memory module 24, and/or a communication module 26. The processor 22 may take any suitable form, such as a microcontroller, plural microcontrollers, circuitry, a single processor, or plural processors configured to execute software instructions. The memory module 24 may take any suitable form or forms, including a volatile memory, such as random access memory (RAM), or non-volatile memory such as read only memory (ROM), flash memory, a hard disk drive (HDD), a solid state drive (SSD), or other data storage media. The memory module 24 may be used by the processor 22 for the temporary storage of data during its operation. Data and software, such as the algorithms or software necessary to perform the methods and provide the functionality discussed herein, as well as an operating system, firmware, or other application, may be installed in the memory module 24. The communication module 26 is arranged to enable communication between the components of the system 10. The communication module 26 may be any module, device, or means capable of enabling the transmission and/or reception of a wired or wireless communication signal, utilizing technologies that include, but are not limited to Wi-Fi (e.g., IEEE 802.11), Bluetooth, cellular, Ethernet, Zigbee, etc.

In the illustrated embodiment, the remote device 15 takes the form of a smartphone, although it is to be appreciated that the remote device 15 may be any computing device capable of data communication (e.g. via the communication module 26) with one or more components of the oral care device 12. For example, the remote device 15 may be, or include, a computer, a laptop, a tablet, a mobile computing device, a server, a gateway, a network node, Internet- or cloud-implemented computing resources, etc. As noted above, the remote device 15 may include its own controller or computing resources that are utilized with computing resources from the oral care device 12, which together comprise the controller 14.

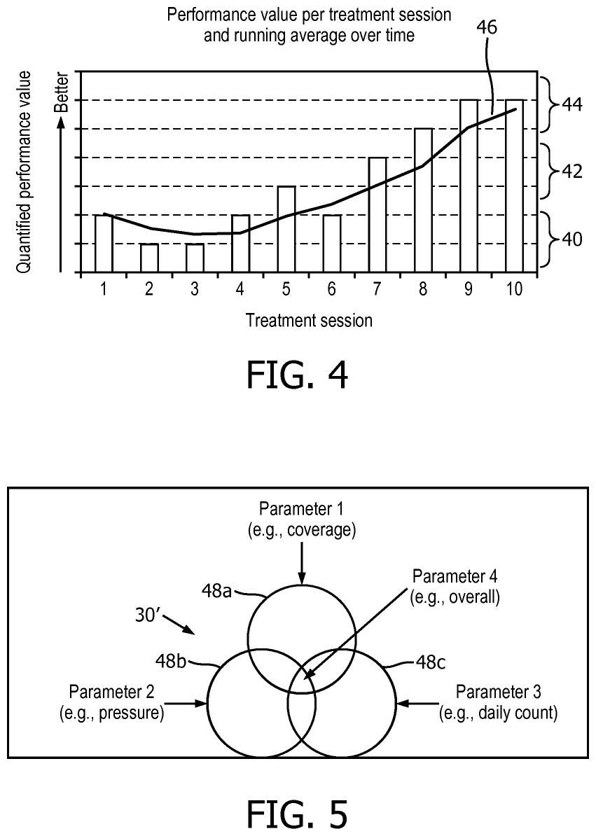

The controller 14 may also include a sensor module 28, which is arranged to collect, during usage sessions while using the oral care device 12, data related to different parameters indicative of a user's oral health. The parameters can be considered “longitudinal” parameters, in that they are to be repeatedly and/or continuously collected over time and stored as historical usage data, e.g., in the memory module 24. The controller 14 uses the collected data (e.g., stored in the memory module 24 and analyzed by the processor 22) to quantify the user's oral health (e.g., determine a numerical value representative of one or more parameters of the user's oral health by comparing the collected data to a set of target, baseline, and/or reference values). Examples for the sensor module 28, the collected data, the parameters and quantified user oral health values are discussed in more detail below, particularly with respect to FIGS. 3-4.

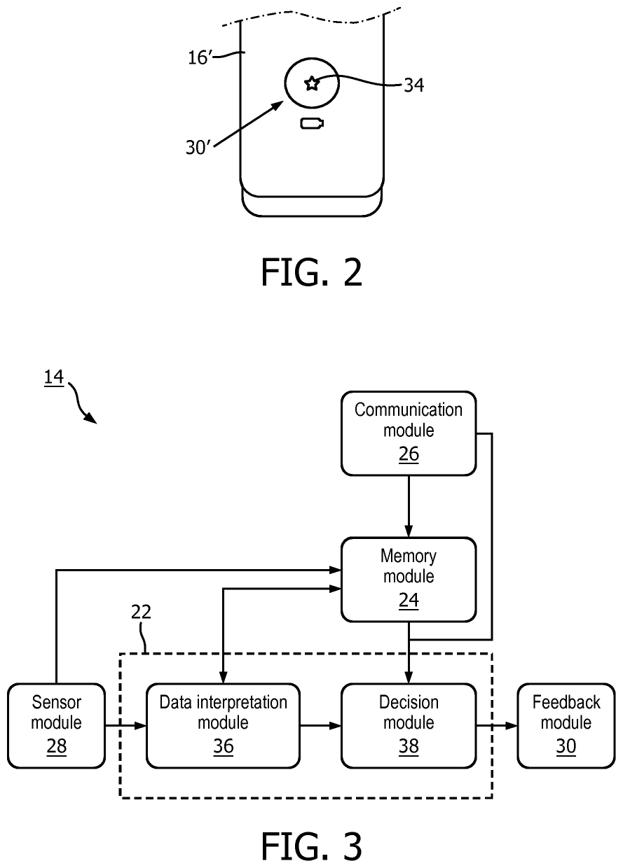

The oral care device 12 may also include a visual feedback module 30 arranged to display some element or feature (e.g., shape, symbol, color, graphic, word, etc.) representative or indicative of the user's quantified oral health values over a period of time, as discussed in more detail below. To this end, the visual feedback module 30 is intended to provide a constant, quickly and easily viewed indication of the user's overall health status or score, as determined by the system 10. For example, the visual feedback module 30 is illustrated in FIG. 1 as including an illuminated portion 32 on the main body 16 of the oral care device 12. For example, the illuminated portion 32 may be selectively illuminated by one or more LEDs or other light sources, in one or more different colors or patterns. The visual feedback module 30 may include a window or other translucent element (e.g., the portion 32 may be arranged as, or with, a window) in the oral care device 12 to facilitate the use of LEDs and other light sources. The visual feedback module 30 may be included in any component of the oral care device 12 that is readily visible by the user, e.g., the main body 16, the charging station 18, the workpiece 20, etc. Alternatively, or additionally, the remote device 15 may include a visual feedback module 30. Those of ordinary skill in the art will appreciate other shapes, sizes, words, colors, graphics, symbols, etc., which can be used in accordance with different embodiments for the visual feedback module 30. For example, FIG. 2 shows an alternate embodiment in which a visual feedback module 30′ of a main body 16′ includes an icon 34 (e.g., shaped like a star) that is arranged to illuminate and/or be illuminated according to the methods discussed herein.

One embodiment for the controller 14 is illustrated in FIG. 3. In this embodiment, the controller 14 includes a memory module 24, a communication module 26, a sensor module 28, a visual feedback module 30, a data interpretation module 36, and a decision module 38. The memory module 24 is discussed above and may be configured to store the data collected by the sensor module 28, including current data (i.e., from the most recent usage session) and historical data (i.e., from previous usage sessions). The communication module 26 is also discussed above and may be utilized by the controller 14 to communicate with any of the other components of the system 10 and/or for the various components of the controller 14 to communicate with each other. One or both of the data interpretation module 36 and the decision module 38 may be implemented by the processor 22. For example, the modules 36 and 38 may include different algorithms that are executed by the processor 22. Alternatively, the modules 36 and/or 38 may be implemented with designated hardware and/or software components included in the controller 14 that are separate from the processor 22.

Ultimately, the purpose of the controller 14 as illustrated in FIG. 3 is to provide a control signal to control operation of the visual feedback module 30. As discussed in more detail below, data collected by the sensor module 28 is stored over time. The stored data (historical and for the current session) is analyzed by the data interpretation module 36 to quantify one or more parameters representative or indicative of the user's oral health when using the oral care device 12 (“oral health values” or “quantified oral health values”), and the decision module 38 compares the quantified oral health values to a set of one or more reference values, and the output is used to generate a control signal. In this way, the operation of the visual feedback module 30 can advantageously be used to quickly convey to the user information regarding the user's overall oral health and/or oral care habits (e.g., whether or not, and/or the degree to which, the user is properly using the oral care device 12, following an oral care regimen with the oral care device 12, etc.). The control signal can be automatically generated after each usage session has ended so that the visual feedback module 30 displays updated feedback for the user of the oral care device 12. Advantageously, the automatic update to the control signal and visual nature of the feedback module 30 enables the oral care device 12 to provide an overview of the user's overall brushing habits or oral health based on the user's typical oral care routine (i.e., without requiring the user to perform extraneous procedures or actions).

The sensor module 28 may include any sensing technology, protocol, type, or modality utilized to collect data related to one or more parameters representative or indicative of the oral health of the user as a result of usage sessions with the oral care device 12. The sensor module 28 may be embedded in a component of the oral care device 12, such as an inertial measurement unit (IMU) or proximity sensor embedded in the main body 16 or the workpiece 20 for measuring movement of the oral care device 12 during use. The sensor module 28 may additionally or alternatively be external to the oral care device, e.g., included by the remote device 15 such as a camera of a smartphone configured to track the position of the oral care device 12 or a user's head during a usage session.

In one embodiment, the sensor module 28 includes a counter that tallies the number of times the oral care device 12 is used, e.g., powered on. In one embodiment, the sensor module 28 includes a clock, timer, or time sensor to collect data related to the duration of use of the oral care device 12. In one embodiment, the sensor module 28 includes a force sensor configured to collect data related to the pressure applied by the oral care device 12 (such as by the workpiece 20) against the user's teeth or tissue. In one embodiment, the sensor module 28 includes a motion sensor (e.g., IMU) or optical sensor (e.g., smartphone camera) for collecting data related to movement of the oral care device 12 and/or the user during a usage session.

The data interpretation module 36 is configured to receive the collected data from the sensor module 28 (e.g., directly, or indirectly from the memory module 24 if the collected data is stored by the memory module 24 after collection). In order for the controller 14 to generate a control signal for the visual feedback module 30 that conveys the user's overall health (e.g., via the user's compliance with an oral care regimen), the data received by the data interpretation module 36 may include historical data, i.e., data corresponding to one or more previous usage sessions. For example, if the collected data is aggregated together, then this aggregated data (or a portion thereof) may be sent over to the data interpretation module 36 and analyzed as a whole. The data (whether aggregated or not) may be timestamped to enable it to be sorted by date and analyzed, e.g., for trends, and/or to enable more recent historical data to be identified. In one embodiment, data older than a certain date is deleted, or only data more recent than a certain date is utilized (e.g., only data from within the past three months, six months, year, or some other time period). In another embodiment, data from each usage session may be associated and a separate interpretation is made by the data interpretation module 36 for each usage session. In one embodiment, the data is associated with respect to individual usage sessions and then the data from multiple usage sessions is received by the data interpretation module 36 and analyzed together.

The data interpretation module 36 is configured to use the collected data to quantify one or more parameters characterizing the user's oral health as a result of the usage sessions over time. The data interpretation module 36 may fuse data from combinations of different sensors to make more accurate or higher level determinations. For example, it may require multiple different sensors to determine the “coverage” of use during the usage session (that is, the locations of the user's mouth the workpiece 20 of the oral care device 12 was moved to, for how long the workpiece 20 was at each location, and at what angle the workpiece 20 of the oral care device 12 was oriented at each location). In one embodiment, coverage of the usage sessions is determined at least in part from the relative position of the oral care device 12 with respect to the user, which can be ascertained by using a movement sensor, such as an embedded IMU, to determine an absolute position of the oral care device 12, and an optical or proximity sensor, such as an infrared proximity sensor or a camera of a smartphone, to account for relative motion of the user's head with respect to the oral care device.

Table 1 below presents examples of data interpretations that can be employed to convert the raw data from the sensor module 28 into usable interpretations (i.e., a quantified value) for a variety of different sensor types. It is to be understood that the examples of Table 1 are not exhaustive and that based on these examples and the disclosure herein, those of ordinary skill in the art will appreciate other data interpretations that can be made with respect to other data types.

TABLE 1

| Sensor Type(s) | Meaning of interpreted data |
|---|---|
| (e.g., for | (e.g., determined by data |
| sensor module 28) | interpretation module 36) |
| Counter | Usage of the device (e.g., number |
| | of times per day, number |
| | of times per week, etc.) |
| Timer | Usage duration of the device per |
| | usage session (e.g., amount of time |
| | that elapsed between the device |
| | turning on and the device turning off) |
| Force sensor | Amount of pressure applied by the |
| (e.g., Hall effect, piezo) | device against tissue (e.g., estimate |
| | applied force/pressure from electric |
| | current generated by piezo element or |
| | deflection measured with Hall effect sensor) |
| Optical sensor | Oxygenation of tissue |
| (e.g., multi wavelength | (e.g., determine hemodynamic |
| fibers) | indices with optical spectroscopy) |
| Motion sensor | Coverage achieved during the usage of |
| (e.g., IMU) | the device (e.g., areas in the user's |
| | mouth that the workpiece was positioned) |
| Motion sensor (e.g., accelerometer) | Amount of scrubbing performed during the |
| and/or Optical sensor | usage of the device (e.g., percentage of |
| (e.g., camera, infrared | time and/or areas in the user's mouth |
| proximity sensor) and/or | that a brush head of an electric toothbrush |
| Force sensor (e.g., Hall effect) | is too closely or firmly pressed against a |
| and/or Timer | user's teeth, which limits the ability |
| | of the bristles to brush) |
| Motion sensor (e.g., IMU) | Coverage achieved during the usage of the device |
| and/or Optical sensor | (e.g., the areas in the user's mouth that the workpiece |
| (e.g., infrared sensor, | is positioned, the angles of the workpiece in the user's |
| camera) and/or Other proximity sensor | mouth at each area, the duration of time at each area, |
| (e.g., ultrasonic) and/or Timer | etc.) |

The decision module 38 is configured to compare the quantified oral health values produced by the data interpretation module 36, as discussed above, to a set of one or more reference values. The set of reference values can be selected as those that are shown, understood, expected, and/or estimated to indicate good oral health of the user. The set of reference values thus may include known or established baselines, thresholds, and/or ranges that the quantified oral health values (as determined by the data interpretation module 36) are expected to align with or otherwise conform to if the user is properly using the oral care device 12. The reference values may be hardcoded into the oral care device 12 or the controller 14 (e.g., in the memory module 24), derived from historical data collected by the sensor module 28, or sent from the remote device 15 and/or otherwise retrieved by the communication module 26. The reference values may be set by the manufacturer of the oral care device 12, by a user or dental health professional, etc.

In one embodiment, the decision module 38 includes a comparator or algorithm that is configured to receive as inputs the set of reference values (e.g., from the memory module 24) and the quantified oral health values (e.g., from the data interpretation module 36), and to compare the quantified oral health values to the set of reference values. In one embodiment, the set of reference values includes one or more ranges and the quantified oral health values are compared to determine whether they fall into each of the corresponding ranges. In one embodiment, the set of reference values includes one or more threshold values and the quantified oral health values are compared to determine whether they are greater and/or less than the threshold values.

In one embodiment the sensor module 28 includes a counter from which the data interpretation module 36 determines the number of times each day that the oral care device 12, e.g., in the form of a toothbrush, is used. In this example, the set of reference values may include a threshold value of ‘2’ for the required number of time per day, and the decision module 38 is thereby configured to compare the value ‘2’ to the quantified oral health value from the data interpretation module 36. The decision module 38 may produce a binary output (e.g., whether or not each quantified oral health value falls into the range delineated by the set of reference values), or a tiered output (e.g., brushing less than two times a day is identified as “poor” and results in the visual feedback module displaying a red light, brushing two times a day is identified as “good” and results in a blue light, brushing three times a day is identified as “great” and results in a green light). Those of ordinary skill in the art will recognize that this is merely one non-limiting example and that the decision module 38 may be used to assess the degree of success or failure of a number of different oral health parameters.

Table 2 is included below to provide additional examples of decisions that can be employed by the controller 14 using the decision module 38 (and which shall also be understood as non-limiting). It is to be appreciated that for each “binary” comparison listed below, multiple binary comparisons for each of a plurality of different ranges of the reference values can be made for each oral health parameter (e.g., a first range indicating “poor” oral health, a second range indicating “adequate” oral health, a third range indicating “good” oral health, etc.).

TABLE 2

| Interpreted Data | Decision Method | Alternative Method |
|---|---|---|
| (e.g., from the data | (e.g., implemented by the | (e.g., implemented by the |
| interpretation module 36) | decision module 38) | decision module 38) |
| Usage of the device | Binary comparison against a | (non)-Linear mapping of usage |
| | predetermined amount per day, | trend, e.g. upward/downward trend, |
| | e.g. 2 sessions per day | flat positive/negative trend |
| Usage duration of the device | Binary comparison against a | (non)-Linear mapping of usage |
| | predetermined amount of time, e.g. | trend, e.g. upward/downward trend, |
| | 2 minutes per session | flat positive/negative trend |
| Amount of pressure applied | Binary comparison against a | (non)-Linear mapping of usage |
| by the device against tissue | predetermined amount of average | trend, e.g. upward/downward trend, |
| | pressure, e.g. less than 300 grams-force | flat positive/negative trend |
| | in average | |
| Amount of scrubbing | Binary comparison against a | (non)-Linear mapping of usage |
| performed during the usage | predetermined amount (time or | trend, e.g. upward/downward trend, |
| of the device | surface area) of scrubbing | flat positive/negative trend |
| | percentage, e.g. less than 5% in | |
| | average | |
| Coverage achieved during | Binary comparison against a | (non)-Linear mapping of usage |
| the usage of the device | predetermined amount of coverage | trend, e.g. upward/downward trend, |
| | percentage, e.g. 90% coverage per | flat positive/negative trend |
| | tooth surface | |
| Oxygenation of tissue | Binary comparison against a | (non)-Linear mapping of usage |
| | predetermined amount of tissue | trend, e.g. upward/downward trend, |
| | oxygenation, e.g. 90% | flat positive/negative trend |
| | oxygenation per tissue area | |

Similar to the data interpretation module 36, in order for the control signal sent to the visual feedback module 30 to represent an overall oral health status for the user, the inputs to the decision module 38 may include quantified oral health values calculated with respect to and/or utilizing historical data (i.e., data collected during one or more previous usage sessions). For example, the decision module 38 may employ any desired statistical model or function (e.g., mean, mode, variance, standard deviation, etc.) to process the historical data and/or to assist in performing the comparison. For example, an average of the quantified oral health values may be compared to the reference values to compute the degree of conformity between the quantified oral health values and the set of reference values. As another example, if the historical data is associated in datasets corresponding to individual usage sessions, then each dataset may be separately processed by the decision module 38. In this example, the control signal may be generated by further processing the results of each dataset/session (e.g., an average, a weighted average favoring more recent usage sessions, a number or percentage of the usage sessions having quantified oral health values that conform sufficiently to the reference values, etc.).

FIG. 4 illustrates a graph according to one embodiment in which the collected historical data has been associated with individual sessions, such that a quantified oral health value is determined for each session over time. In the example embodiment of FIG. 4, quantified oral health values for ten sessions are illustrated, although it is to be appreciated that data associated with any other number of usage sessions could be collected, stored, and analyzed. In this example, a first range 40, a second range 42, and a third range 44 for the quantified oral health values are illustrated, and which may correspond to different ranges for the reference values as discussed herein. For example, the first range 40 may define reference values that are expected to indicate “poor” oral health, the second range 42 may define reference values that are expected to indicate “adequate” oral health, and the third range 44 may define reference values that are expected to indicate “good” oral health. Thus, in the example of FIG. 4, the plotted data indicates that the user has generally improved their oral health over time.

A line 46 depicted on the graph of FIG. 4 to represent a running average of the quantified oral health values over time. In accordance with the above description, the value of the running average depicted by the line 46 can be compared by the decision module 38 to the reference values (e.g., the ranges 40, 42, and 44) to generate the control signal after each of the usage sessions. In this example, the running average has a period of three after the third usage session (i.e., it is calculated as the average of the last three usage sessions), although any other number of periods could be utilized for calculating running averages. Furthermore, as noted above, any other type of data processing (e.g., statistical function) may be implemented to simplify the collected data and/or the quantified oral health values associated with a plurality of different usage sessions, into a form that facilitates comparison to the reference values by the decision module 38 (e.g., average of all historical data, weighted average, the number or percentage of sessions that have oral health values that conform to a sufficient degree when compared to the reference values, etc.). Thus, it is to be appreciated that the running average depicted by the line 46 is one specific, non-limiting example of data processing that can be performed (e.g., by the data interpretation module 36) to facilitate operation of the decision module 38 and/or generation of the control signal for the visual feedback module 30.

More specifically with respect to the example of FIG. 4, the value of the running average after the first, second, third, and fourth sessions would be determined by the decision module 38 as falling into the first range 40, and therefore, the control signal sent to visual feedback module 30 after each of these sessions would instruct the visual feedback module 30 to operate in accordance with a first set of operational characteristics corresponding to the first range 40. Similarly, the control signal after the fifth, sixth, seventh, and eighth usage sessions would instruct operation in accordance with a second set of operational characteristics since the value of the running average after each of these sessions would be determined by the decision module 38 as falling within the range 42. Lastly, the control signal after the ninth and tenth usage sessions would instruct operation of the visual feedback module 30 in accordance with a third set of operational characteristics, since the decision module 38 would determine the value of the running average after these sessions to fall within the third range 44. As noted above, the different sets of operational characteristics may correspond to different colors (e.g., red, yellow, green as the oral health improves through each of the ranges 40, 42, and 44). In another example, additional symbols or segments of a symbol may be illuminated as each of the ranges is met (e.g., a bar having multiple segments may be increasingly illuminated as the oral health improves, with a fully-illuminated bar indicating good oral health and an unilluminated bar indicating poor oral health).

It is to be appreciated that trends in the user's oral health can also, or alternatively, be assessed by the decision module 38. For example, the third column of Table 2 includes some linear or non-linear determinations that can be made to assess trends. As another example, with reference to the example of FIG. 4, the slope of the line 46 may be determined after each usage session in order to track the trend in the user's oral health over time. In one embodiment, the decision module 38 is configured to assess whether the slope is positive (e.g., after any of the sessions five through ten in FIG. 4) or negative (e.g., after the second or third sessions). In another embodiment, the value from each most recent usage session is compared to one or more previous usage sessions (e.g., the difference between session six and session five in FIG. 4 being negative, while the difference between session seven and session six being positive). For example, trends that indicate improved oral health (e.g., positive slopes) may result in the visual feedback module 30 being operated with a “positive” set of characteristics, such as a green light, thumbs up symbol, etc., while trends that indicate worsening oral health (e.g., negative slopes) may result in the visual feedback module 30 being operated with a “negative” set of characteristics, such as a red light, thumbs-down symbol, etc.

The output from the decision module 38 is used to generate a control signal (e.g., created by the decision module 38, the processor 22, or other component of the controller 14) that is sent to and controls operation of the visual feedback module 30. In discussed above, the visual feedback module 30 may include a light source embedded in the oral care device 12, e.g., with a light path viewable by users in the ambient environment through, or in the form of, a geometric shape, symbol, word, pattern, etc.

The visual feedback module 30 may be operated with different operational characteristics depending on the degree of conformity determined by the decision module 38 when comparing the quantified oral health values to the set of reference values. For example, if the set of reference values includes one or more ranges and the quantified oral health values fall within those ranges, then the visual feedback module 30 may be operated with a first set of operational characteristics (e.g., illuminate in a first color, such as green, or display a symbol, such as a star, thumbs-up, smiley face, etc.), while if the quantified oral health values fall outside of the set ranges, then the control signal may result in the feedback module 30 being operated with a second set of operational characteristics (e.g., to not illuminate, illuminate in a second color, such as red, or display a symbol such as a thumbs-down or frowny face). In one embodiment, particularly where binary decisions are made by the decision module 38, at least one element of the visual feedback module 30 is turned on or off (e.g., a different number of LEDs are powered, a different number of segments or symbols of a display are illuminated, etc.). In other embodiments, particularly where (non)-linear decisions are made by the decision module 38, the control signal for the visual feedback module 30 may be assigned a continuous value of the output scale.

It may be desirable in some embodiments to provide feedback for more than one oral health parameter. To this end, the visual feedback module 30 may include a combination of different elements (e.g., light sources, windows, segments) in the oral care device 12 that enable multiple light paths from the oral care device 12 to be viewed by users. An example is depicted in FIG. 5, which illustrates a visual feedback module 30′ that has a plurality of elements 48a, 48b, and 48c. Each of the elements 48a, 48b, and 48c may be separately controlled (e.g., illuminated) and associated with a different oral health parameter. For example, each of the elements 48a-48c may be light sources operated at different colors, intensities, etc. depending on the degree of conformity of the user's quantified oral health values to the corresponding reference values for each of the parameters. In this example, the element 48a may correspond to a coverage parameter (or other first parameter), the element 48b may correspond to a pressure parameter (or other second parameter), and the element 48c may correspond to a daily count parameter (or other third parameter), each of which parameters are discussed above. From the combination of parameters, a final parameter representative of the overall health for the user can be understood. Advantageously, the visual feedback module 30′ enables a user to receive feedback for each of the selected parameters independently, as well as the overall oral health by considering all parameters at once.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. As used herein in the specification and in the claims, “or” should be understood to have the same meaning as “and/or” as defined above.

It should also be understood that, unless clearly indicated to the contrary, in any methods claimed herein that include more than one step or act, the order of the steps or acts of the method is not necessarily limited to the order in which the steps or acts of the method are recited.

1. An oral care system comprising:
an oral care device;
a sensor module configured to collect data during usage sessions with the oral care device related to one or more longitudinal parameters representative of oral health of a user;
a memory module configured to store the data;
a data interpretation module configured to retrieve historical usage data from the memory module and quantify one or more oral health values from the historical usage data with respect to each of the one or more longitudinal parameters;
a decision module configured to compare the one or more health values to a set of one or more reference values; and
a controller configured to generate a control signal based on an output of the decision module; and
a visual feedback module of the oral care device configured to operate in accordance with the control signal.

2. The system of claim 1, wherein the one or more longitudinal parameters includes a plurality of longitudinal parameters, the one or more oral health values includes a plurality of oral health values, and the data interpretation module is configured to quantify one of the oral health values for each of the longitudinal parameters.

3. The system of claim 2, wherein the controller is configured to generate a plurality of control signals, with one of the control signals generated with respect to each of the oral health values, and the visual feedback module includes separate elements that are independently controllable with each of the control signals.

4. The system of claim 1, wherein the visual feedback module includes one or more light sources.

5. The system of claim 4, wherein the control signal instructs the one or more light sources to be operated with a first set of operating characteristics if the output of the decision module relates to a sufficient degree of conformity of the one or more oral health values to the set of one or more reference values and to be operated with a second set of operating characteristics if the output of the decision module relates to an insufficient degree of conformity of the one or more oral health values to the set of one or more reference values.

6. The system of claim 5, wherein the first and second operating characteristics include different colors, patterns, intensities, number of illuminated light sources, or a combination including at least one of the foregoing.

7. The system of claim 1, wherein the set of reference values includes at least one threshold value or range of values.

8. The system of claim 1, wherein data collected during each usage session is arranged in a dataset associated with that usage session.

9. The system of claim 8, wherein the one or more oral health values comprises a plurality of oral health values, and the controller is configured to quantify one of the oral health values for each of the datasets.

10. The system of claim 1, wherein the historical usage data includes a most recent usage session and at least one previous usage session aggregated together.

11. The system of claim 1, further comprising a remote device in communication with the oral care device, the remote device at least partially comprising the controller.

12. The system of claim 1, wherein the oral care device includes a main body, a workpiece, a charging station, or a combination including at least one of the foregoing.

13. An oral care device, comprising:
a sensor module configured to collect data during usage sessions with the oral care device related to a longitudinal parameter representative of oral health of a user;
a memory module configured to store the data;
a controller configured to, after each usage session of the oral care device, automatically:
retrieve the data for a most recent usage session and at least one previous usage session;
quantify an oral health value of the longitudinal parameter from the retrieved data;
perform a comparison of the oral health value to a set of one or more reference values; and
generate a control signal based on the comparison; and
a visual feedback module of the oral care device configured to operate in accordance with the control signal.

14. A method for providing visual feedback to a user of an oral care device, comprising:
collecting data during a usage session of the oral care device, the data related to a longitudinal parameter representative of oral health of the user;
storing the data in a memory module;
retrieving historical usage data from the memory module;
quantifying an oral health value of the longitudinal parameter from the retrieved data;
comparing the oral health value to a set of one or more reference values;
generating a control signal based on the comparing; and
operating a visual feedback module of the oral care device in accordance with the control signal;
wherein one or more of the receiving, quantifying, comparing, and generating occur automatically after usage sessions of the oral care device.

15. The method of claim 14, wherein the generating includes generating a plurality of control signals, the visual feedback module includes a plurality of separate elements, and the operating includes separately controlling each of separate elements of the visual feedback module in accordance with a different one of the control signals.
